(19) United States
(12) Patent Application Publication
(10) Pub. No.: US 2024/0024687 A1
(43) Pub. Date: Jan. 25, 2024

(54) SENSORY STIMULATION

(71) Applicant: MAGNES AG, Zurich (CH)

(72) Inventors: Alessandro SCHAER, Zurich (CH); Barna Errol Mario BECSEK, Zurich (CH); Tayfun TATAR, Zurich (CH); Georgios CHATZIPIRPIRIDIS, Zurich (CH); Olgac ERGENEMAN, Zurich (CH)

(21) Appl. No.: 18/254,175

(22) PCT Filed: Nov. 25, 2021
(86) PCT No.: PCT/EP2021/082963

§ 371 Date: May 24, 2023

(30) Foreign Application Priority Data
Nov. 26, 2020 (GB) 2018645.8

(51) Int. Cl. A61N 1/365; A61B 5/11; A61B 5/00

(52) U.S. Cl. CPC A61N 1/36542; A61B 5/112; A61B 5/6807; A61B 5/1117; A61B 5/7267; A61B 5/1112; A61B 2562/0219; A61B 2562/0271

(57) ABSTRACT

A system for applying sensory stimulation to a subject's foot or ankle based on detected gait kinematics for therapy, training, or movement assistance. The system comprises at least one item of footwear incorporating one or more sensors, one or more vibration actuators, a data processor, memory, and a data transceiver. The system further comprises a remote computing system comprising data communication means and data processing means. The sensors are configured, during a calibration phase, to generate calibration sensor data associated with movement of a subject wearing the item of footwear. The data transceiver is configured to communicate the calibration sensor data to the remote computing system. The remote computing system is configured to receive, via the data communication means, the calibration sensor data from the item of footwear. The remote computing system is further configured to process, using the data processing means, the calibration sensor data to: generate one or more gait parameters associated with the subject's gait kinematics; generate, using the gait parameters and one or more program parameters associated with a program of therapy, training, or movement assistance, a predicted distribution of sensor data values within which further sensor data generated by the one or more sensors is predicted to fall in the event of movement of the subject requiring sensory stimulation to be applied in accordance with the program of therapy, training or movement assistance. The remote computing system is configured to communicate, via the data communication means, the predicted distribution of sensor data values to the data transceiver. The data transceiver is configured to receive the predicted distribution of sensor data values and said memory is configured to store the predicted distribution of sensor data values. Whereupon, during an operational phase the data processor is configured to monitor further sensor data from the one or more sensors, and, in the event that the further sensor data from the one or more sensor data falls within the predicted distribution of sensor data values, the data processor is configured to control the

one or more vibration actuators to provide sensory stimulation in accordance with the program of therapy, training or movement assistance.

### TECHNICAL FIELD

The present invention relates to techniques for generating sensory stimulation for stimulating a subject's foot for therapy, training, or movement assistance.

### BACKGROUND

Techniques for stimulating human feet with vibration for therapeutic reasons are known in the art (see for example “Subsensory vibrations to the feet reduce gait variability in elderly fallers”, Galica et al).

Typically, these techniques involve monitoring certain aspects of a subject's gait kinematics to recognise movement requiring vibrating stimulation to be applied, and then applying the relevant vibration.

Often these techniques are used in a motion analysis laboratory. However, certain systems have been proposed that can be used outside of a laboratory setting.

WO2017/023864 for example proposes a system for alleviating knee osteoarthritis by modifying a subject's gait kinematics using electrical or vibrotactile sensory stimulation applied to the subject's foot.

A device is proposed that includes sensors and a processor which are incorporated in a shoe or band worn by the subject and which detects sensor signals associated with a subject's motion. These sensor signals are then processed by the processor to identify gait parameters associated with the gait kinematics of the subject which are then in turn used to control the application of the sensory stimulation.

### SUMMARY OF THE INVENTION

In accordance with a first aspect of the invention, there is provided a system for applying sensory stimulation to a subject's foot or ankle based on detected gait kinematics for therapy, training, or movement assistance. The system comprises at least one item of footwear incorporating one or more sensors, one or more vibration actuators, a data processor, memory, and a data transceiver. The system further comprises a remote computing system comprising data communication means and data processing means. The sensors are configured, during a calibration phase, to generate calibration sensor data associated with movement of a subject wearing the item of footwear. The data transceiver is configured to communicate the calibration sensor data to the remote computing system. The remote computing system is configured to receive, via the data communication means, the calibration sensor data from the item of footwear. The remote computing system is further configured to process, using the data processing means, the calibration sensor data to: generate one or more gait parameters associated with the subject's gait kinematics; generate, using the gait parameters and one or more program parameters associated with a program of therapy, training, or movement assistance, a predicted distribution of sensor data values within which further sensor data generated by the one or more sensors is predicted to fall in the event of movement of the subject requiring sensory stimulation to be applied in accordance with the program of therapy, training or movement assistance. The remote computing system is configured to communicate, via the data communication means, the predicted distribution of sensor data values to the data transceiver. The data transceiver is configured to receive the predicted distribution of sensor data values and said memory is configured to store the predicted distribution of sensor data values. Whereupon, during an operational phase the data processor is configured to monitor further sensor data from the one or more sensors, and, in the event that the further sensor data from the one or more sensor data falls within the predicted distribution of sensor data values, the data processor is configured to control the one or more vibration actuators to provide sensory stimulation in accordance with the program of therapy, training or movement assistance.

Optionally, during a further calibration phase, the sensors are configured to generate further calibration sensor data associated with subsequent movement of the subject and said data transceiver is configured to communicate the further calibration sensor data to the remote computing system. The remote computing system is configured to receive, via the data communication means, the further calibration sensor data, process, using the data processing means, the further calibration sensor data to: generate one or more updated gait parameters associated with the subject's gait kinematics; generate, using the updated gait parameters and one or more program parameters associated with a program of therapy, training, or movement assistance, an updated predicted distribution of sensor data values within which the further sensor data generated by the one or more sensors is predicted to fall in the event of movement of the subject requiring sensory stimulation to be applied in accordance with the program of therapy, training or movement assistance, said remote computing system configured to communicate, via the data communication means, the updated predicted distribution of sensor data values to the data transceiver. The data transceiver is configured to receive the updated predicted distribution of sensor data values and said memory is configured to store the updated predicted distribution of sensor data values. Whereupon, during a further operational phase the data processor is configured to monitor the further sensor data from the one or more sensors, and, in the event that the sensor data from the one or more sensor data falls within the updated predicted distribution of sensor data values, said data processor is configured to control the one or more vibration actuators to provide sensory stimulation in accordance with the program of therapy, training or movement assistance.

Optionally, the data processing means is configured to periodically generate the updated predicted distribution of sensor data values at a predetermined interval and/or responsive to an update signal from the remote computing system.

Optionally, the gait parameters include one or more of gait speed, step velocity, step length, swing time variability, stride length, step width, rhythm, variability, asymmetry, postural control and step characteristics.

Optionally, the one or more vibration actuators are configured to generate sub-sensory vibration.

Optionally, the item of footwear incorporates a plurality of vibration actuators.

Optionally, the data processor is configured to actuate each vibration actuator to generate foot stimulating vibration at a vibration level determined during a calibration process in which a subject's sensory perception is assessed at each foot position corresponding to a position of each vibration actuator thereby accommodating differences in sensory perception across the subject's foot.

Optionally, the vibration level comprises a predetermined vibration frequency and/or predetermined vibration amplitude.

Optionally, the sensory stimulation is tactile cuing.

Optionally, the one or more vibration actuators are embedded in a sole or insole of the at least one item of footwear.

Optionally, the one or more vibration actuators are embedded in the sole or insole of the at least one item of footwear such that, in use, vibrations are transferred to the subject's foot via an intermediate portion of the sole or insole separating each of the one or more vibration actuators and the subject's foot.

Optionally, the one or more sensors, data processor, memory and data transceiver are also embedded in the sole or insole of the item of footwear.

Optionally, the sensors comprise one or more inertial measurement units comprising one or more of an accelerometer, gyroscope, and magnetometer.

Optionally, the sensors further comprise one or more of a foot-pressure sensor for detecting pressure changes arising due to the subject contacting the ground, a temperature sensor for detecting an ambient temperature, a barometric pressure sensor for detecting barometric pressure and a sound sensor.

Optionally, the at least one item of footwear further incorporates movement distance tracking means configured to generate movement distance data associated with a distance moved by the item of footwear and the remote computing system has running thereon a movement distance analysis function. The data transceiver is configured to communicate the movement distance data to the remote computing system for analysis by the movement distance analysis function to generate movement distance analysis data.

Optionally, the program of therapy, training or movement assistance is a program of movement assistance for alerting a subject to a potential fall, said predicted distribution of sensor data values corresponding to a range of sensor values predicted to arise in the event the subject's gait kinematics change in such a way indicating an imminent fall, said data processor thereby operable to control the one or more vibration actuators to generate sensory stimulation in the event of an imminent fall being detected.

Optionally, the program of therapy, training or movement assistance is a program of therapy for alerting a subject to a potential episode of gait freeze, said predicted distribution of sensor data values corresponding to a range of sensor values predicted to arise in the event the subject's gait kinematics change in such a way indicating an imminent episode of gait freeze, said data processor thereby operable to control the one or more vibration actuators to generate sensory stimulation in the event of an imminent episode of gait freeze detected.

Optionally, the program of therapy, training or movement assistance is a program of training for alerting a subject to movement that deviates from a desired movement form, said predicted distribution of sensor data values corresponding to a range of sensor values predicted to arise in the event the subject's gait kinematics change in such a way indicating that the desired movement form has been deviated from, said data processor thereby operable to control the one or more vibration actuators to generate sensory stimulation in the event of a desired movement form has been deviated from.

Optionally, the program of therapy, training or movement assistance is a program of training for alerting a subject to movement that is in accordance with a desired movement form, said predicted distribution of sensor data values corresponding to movement of the subject in the event the subject's gait kinematics indicate that a desired movement form is being maintained, said one or more vibration actuators thereby providing sensory stimulation in the event of a desired movement form is maintained.

Optionally, the at least one item of footwear comprises a rechargeable battery for powering the components incorporated therein.

Optionally, the at least one item of footwear comprises a single vibration actuator positioned to stimulate a foot position of a subject at one of the first metatarsophalangeal joint, the fifth metatarsophalangeal joint, the heel, the medial longitudinal arch, the big toe, the ankle or the upper part of foot.

Optionally, the at least one item of footwear comprises a plurality of vibration actuators positioned to stimulate foot positions of a subject at one or more of the first metatarsophalangeal joint, the fifth metatarsophalangeal joint, the heel, the medial longitudinal arch, the big toe the ankle and the upper part of foot.

In accordance with a second aspect of the invention, there is provided a method of applying sensory stimulation to a subject's foot or ankle based on detected gait kinematics for therapy, training, or movement assistance, said method comprising, during a calibration phase: generating at an item of footwear calibration sensor data associated with movement of a subject wearing an item of footwear; communicating the calibration sensor data from the item of footwear to a remote computing system, generating, at the remote computing system, using the calibration sensor data, one or more gait parameters associated with the subject's gait kinematics; generating, at the remote computing system, using the gait parameters and one or more program parameters associated with a program of therapy, training, or movement assistance, a predicted distribution of sensor data values within which further sensor data generated at the item of footwear is predicted to fall in the event of movement of the subject requiring sensory stimulation to be applied in accordance with the program of therapy, training or movement assistance; communicating the predicted distribution of sensor data values to the item of footwear, and, during an operational phase: monitoring further sensor data generated at the item of footwear, and, in the event that the further sensor data falls within the predicted distribution of sensor data values, controlling one or more vibration actuators to provide sensory stimulation in accordance with the program of therapy, training or movement assistance.

In accordance with a third aspect of the invention, there is provided an arrangement for fitting to an item of footwear for use in a system according the first aspect. The arrangement comprises one or more sensors, one or more vibration actuators, a data processor, memory, and a data transceiver. The sensors are configured, during a calibration phase, to generate calibration sensor data associated with movement of a subject wearing the item of footwear and said data transceiver is configured to communicate the calibration sensor data to a remote computing system, and said data transceiver is configured to receive a predicted distribution of sensor data values from the remote computing system and said memory is configured to store the predicted distribution of sensor data values. Whereupon, during an operational phase said data processor is configured to monitor further sensor data from the one or more sensors, and, in the event that the further sensor data from the one or more sensor data falls within the predicted distribution of sensor data values, said data processor is configured to control the one or more vibration actuators to provide sensory stimulation in accordance with the program of therapy, training or movement assistance.

In accordance with a fourth aspect of the invention, there is provided an item of footwear fitted to which is an arrangement according to the third aspect of the invention.

In accordance with a fifth aspect of the invention, there is provided a pair of items of footwear, comprising a left hand item of footwear according to the fourth aspect of the invention and a right hand item of footwear according to the fourth aspect of the invention.

In accordance with a sixth aspect of the invention, there is provided a computer program for running on a data processor incorporated in an item of footwear and for use in a system according to the first aspect. The computer program comprises computer implementable instructions which when implemented on a data processor, controls the data processor to perform a method comprising: generating at the item of footwear calibration sensor data associated with movement of a subject wearing an item of footwear; communicating the calibration sensor data from the item of footwear to a remote computing system; receiving, from the remote computing system a predicted distribution of sensor data values to the item of footwear, and, during an operational phase: monitoring further sensor data generated at the item of footwear, and, in the event that the further sensor data falls within the predicted distribution of sensor data values, and controlling one or more vibration actuators to provide sensory stimulation in accordance with the program of therapy, training or movement assistance.

In accordance with a seventh aspect of the invention, there is provided a computer program for running on a data processing means of a remote computing system for use in a system according to the first aspect. The computer program comprises computer implementable instructions which when implemented on a data processing means, controls the data processor to perform a method comprising: receiving calibration sensor data from an item of footwear; generating, using the calibration sensor data, one or more gait parameters associated with a subject's gait kinematics; generating, using the gait parameters and one or more program parameters associated with a program of therapy, training, or movement assistance, a predicted distribution of sensor data values within which further sensor data generated at the item of footwear is predicted to fall in the event of movement of the subject requiring sensory stimulation to be applied in accordance with the program of therapy, training or movement assistance, and communicating the predicted distribution of sensor data values to the item of footwear.

In accordance with embodiments of the invention a system for applying sensory stimulation to a subject's feet or ankles based on detected gait kinematics is provided for the purpose of therapy or training which has an optimised system architecture.

Typically, a pair of items of footwear are provided which each include a plurality of sensors, one or more vibration actuators, a data processor and corresponding memory and a data transceiver. The plurality of sensors are provided with each item of footwear and are configured to detect movement of the subject and generate associated sensor data. This sensor data is then transmitted to a remote computing system for analysis and in particular to characterise aspects of the subject's gait. Once the subjects gait has been characterised in this way, the remote computing system is configured to predict sensor data values, based on the subject's gait that would be generated in the event that the subject has moved in such a way that requires sensory stimulation to be applied in accordance with a therapy program or training program. These predicted sensor data values are then communicated back to the data processor on each item of footwear. The data processor on each item of footwear is configured to monitor the sensor data generated by the plurality of sensors and if the sensor data corresponds with the predicted sensor data values, then the data processor can control the vibration actuator (or vibration actuators) to vibrate and therefore generate the required sensory stimulation.

In accordance with examples of the invention, it has been recognised that gait characterising processing tasks associated with generating sensory stimulation to a subject's feet based on detected gait kinematics, whilst more processor intensive, do not necessarily need to be undertaken in “real-time”.

Accordingly, using modern data communication techniques, these tasks can be performed away from where the sensor data is detected on a remote computing device (where there are fewer constraints on power consumption and processor capability). This means the data processing requirements of the data processor located with the sensors can be reduced and need only be capable of matching sensor signals with predicted sensor data values provided by the remote computing system indicating that sensory stimulation should be generated.

Moreover, by undertaking the gait characterising processing tasks at a remote computing device, more complicated gait analysis can be undertaken than would not typically be possible if the gait characterising processing tasks were being performed on the local data processor located with the sensors. Further, sensor data from two items of footwear worn as a pair by the subject can be readily processed to generate more accurate and detailed gait characterising information than would be possible if the gait characterising processing tasks were being performed individually and independently by the data processes located at each item of footwear.

In accordance with embodiments of the invention, data relating to a subject can be collected outside the clinical environment, for example in a familiar setting with unbiased conditions which is likely to lead to intrinsically better analyses and associated therapies. In accordance with embodiments of the invention, a subject's gait can be analysed quantitatively, objectively and in a reproducible manner. In certain applications it is possible for a therapist, for example, to determine in an objective and unbiased way, if a patient has progressed.

Various further features and aspects of the invention are defined in the claims.

### BRIEF DESCRIPTION OF THE DRAWINGS

Embodiments of the present invention will now be described by way of example only with reference to the accompanying drawings where like parts are provided with corresponding reference numerals and in which:

FIG. 1a provides a simplified schematic diagram of a system arranged in accordance with certain embodiments of the invention;

FIG. 1b provides a simplified schematic diagram of a sensory stimulation unit arranged in accordance with certain embodiments of the invention;

FIG. 2 provides a flow chart depicting the operation of the system shown in FIG. 1a

FIG. 3 provides a simplified schematic diagram of the components of a sensory stimulation unit for fitting to an item of footwear in accordance with certain embodiments of the invention;

FIG. 4 provides a simplified schematic diagram depicting the sensors of a sensor unit in accordance with certain embodiments of the invention;

FIG. 5 provides a simplified schematic diagram depicting the sensors of a further sensor unit in accordance with certain embodiments of the invention;

FIG. 6 provides a diagram depicting the location of vibration actuators in accordance with certain embodiments of the invention;

FIG. 7 provides a simplified schematic diagram depicting the incorporation of a sensory stimulation unit in a modified shoe sole in accordance with certain embodiments of the invention;

FIG. 8 provides a simplified schematic diagram of the components of a further sensory stimulation unit for fitting to an item of footwear in accordance with certain embodiments of the invention, in particular further including a location tracking device, and

FIG. 9a provides a simplified schematic diagram depicting an embodiment of the invention in which a sensory stimulation unit is incorporated in an item of footwear to apply sensory stimulation to a subject's ankle, and

FIG. 9b provides a simplified schematic diagram depicting an embodiment of the invention in which a sensory stimulation unit is incorporated in an item of footwear to apply sensory stimulation to an upper side of a subject's foot.

### DETAILED DESCRIPTION

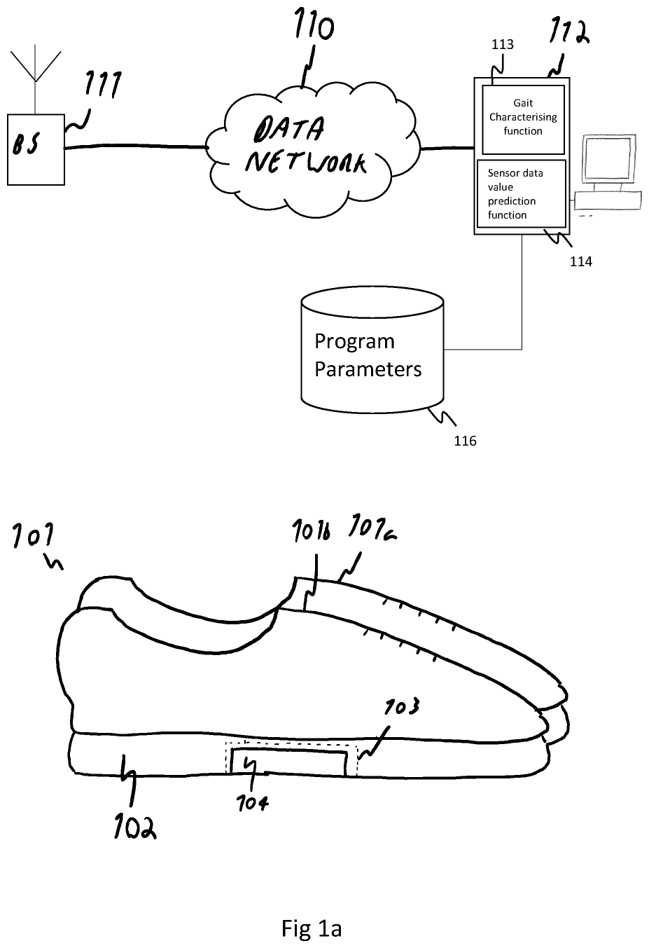

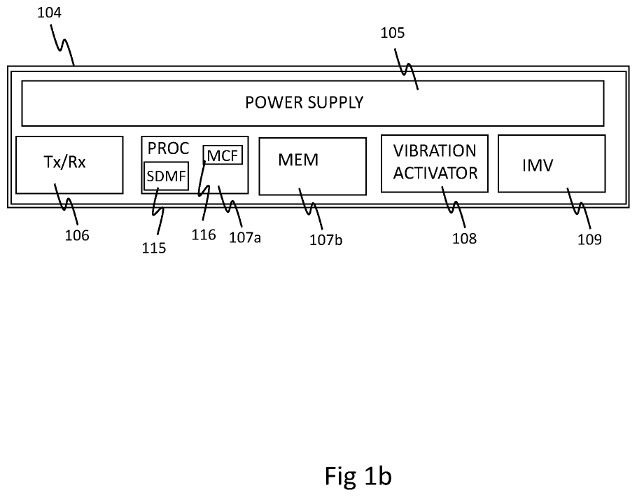

FIG. 1a provides a schematic diagram of a system for applying sensory stimulation to a subject's foot in accordance with certain embodiments of the invention.

The system comprises a pair of items of footwear provided by a pair of shoes 101 comprising a first shoe 101a and a second shoe 101b. Typically, the first shoe 101a and second shoe 101b, other than being configured to fit on the subject's right and left foot respectively, are otherwise identical.

The sole 102 of each shoe 101a, 101b, comprises a cavity 103 within which is mounted a sensory stimulation unit 104.

FIG. 1b provides a simplified schematic diagram providing a more detailed view of the components of the sensory stimulation unit 104.

The sensory stimulation unit 104 comprises a power supply unit 105, a wireless communication unit 106, a data processor 107a and corresponding memory unit 107b, a vibration actuator 108 and a sensor unit 109 comprising a plurality of sensors.

The power supply unit 105 may be provided by a suitable rechargeable battery as is known in the art. The battery may be recharged by any suitable means, for example by a suitable power cable input interface or by inductive coils incorporated in the power supply for wireless charging.

The system further comprises a data network 110 and a wireless base station 111 via which the sensory stimulation unit 104 is configured to transmit data to, and receive data from, a remote computing system 112.

In certain examples, the remote computing system 112 is provided by one or more suitably programmed remote application servers. The data network 110 can be provided by any suitable network for transmitting data between computing devices, for example the internet. The wireless base station 111 can be provided by any suitable wireless access point, that is compatible with the wireless communication unit 106, and is suitable for enabling data to be communicated to and received from the data network 110 for example a suitably connected Wi-Fi router. In alternative embodiments, the wireless base station 111 could be provided by a smart phone, a similar mobile device, a tablet, or any other device with the appropriate communication functionality.

In use, during a calibration phase, for each sensory stimulation unit 104 in each shoe, the plurality of sensors of the sensor unit 109 are configured to detect the movement of the subject when wearing the shoe and generate corresponding sensor data associated with this movement. Under the control of the data processor 107a, this sensor data is then communicated to the remote computing system 112 via the wireless base station 111 and data network 110.

Typically, this sensor data comprises at least one or more of linear acceleration data (generated by an accelerometer), angular velocity data (generated by a gyroscope) and orientation data (generated by a magnetometer).

The remote computing system 112 has running thereon a gait characterising function 113 which is configured to process the sensor data from the sensory stimulation unit of each shoe 101a, 101b to characterise aspects of the subject's gait kinematics. Advantageously, by receiving sensor data from both shoes 101a, 101b, the gait characterising function 113 can more accurately characterise aspects of the subject's gait because sensor data relating to the movement of each foot is generated independently.

The gait characterising function 113 implements one or more gait characterising algorithms which receive as input the sensor data and, from this, generates one or more specific gait parameters associated with the subject's gait as derivable from the sensor data. Techniques for converting such sensor data into gait parameters are well known. For example, it is well known to use peaks, valleys, and zero/crossings in sensor data generated by sensors monitoring human movement to identify “gait events” such as toe-off and heel-strike and so on.

The gait parameters generated by the gait characterising algorithm or gait characterising algorithms can include any one of, or any combination of more than one of: gait speed, step velocity, step length, swing time variability, stride length, step width, rhythm (such as step time, swing time, stance time, single support, double support), variability (such as step velocity variability, step length variability, step time variability, stance time variability), asymmetry (such as swing time asymmetry, step time asymmetry, stance time asymmetry), postural control (such as step length asymmetry), step characteristics (strike angle, minimum toe clearance, foot angles (such as supination angle, strike angle, lift-off angle, angular velocity), peak parameters (such as peak propulsion, peak braking), force/pressure values and power. The gait parameters may further include one or more of loading intensity and cycle and pressure distribution.

The remote computing system 112 also has running thereon a sensor data value prediction function 114. The sensor data value prediction function 114 is configured to receive the gait parameters from the gait characterising function 113. The sensor data value prediction function 114 is further configured to receive program parameters specified by a program of therapy, program of movement assistance or program of training from a program parameter database 117 connected to the remote computing device. These program parameters quantify how aspects of a subject's gait kinematics will change from their normal movement, in the event that intervention, in the form of sensory stimulation is required.

Using one gait parameter, a combination of gait parameters or all the gait parameters and one or more program parameters specified by a program of therapy, program of movement assistance or program of training, the sensor data value prediction function 114 is configured to generate a predicted distribution of sensor data values within which the sensor data generated by the sensor unit 109 would fall in the event that the subject's gait kinematics change in a way requiring stimulating vibration to be applied in accordance with the parameters specified by a program of therapy, program of movement assistance or program of training. The distribution of sensors values typically includes absolute measurement values, and relative timings of these absolute measurement values.

Once the sensor data value prediction function 114 has generated a predicted distribution of sensor data values in this way, the remote computing system 112 is configured to communicate the predicted distribution of sensor data values to the sensory stimulation unit via the data network 110 and the wireless base station 111.

On receipt of the predicted distribution of sensor data values, the data processor 107a is configured to store predicted distribution of sensor data values in the memory unit 107b.

The data processor 107a has running thereon a sensor data monitoring function 115 which is configured, during an operational phase, to monitor the sensor data received from the sensor unit 109 and monitor whether or not the sensor data generated by the sensor unit 109 falls within the predicted distribution of sensor data values.

In the event that the sensor data monitoring function 115 determines that the sensor data generated by the sensor unit 109 falls within the predicted distribution of sensor data values, a gait event detected signal is communicated to a motor control function 116 also running on the data processor 107a. The manifestation of the “gait event detected signal” communicated from the sensor data monitoring function 115 to the motor control function 116 will depend on the implementation of the system. In embodiments in which the sensor data monitoring function 115 and motor control function 116 are implemented as software modules or firmware modules running on the data processor 107a, the gait event detected signal will be in the form of a suitable data exchange (or “message”) of the type known different software components or firmware components running on a data processor communicate data with each other.

The motor control function 116 then controls the data processor 107a to generate, based on the gait event detected signal, an appropriate control signal which is sent to the vibration actuator 108 causing an appropriate stimulation to be applied to the subject's foot.

Typically, vibrations are transferred to the subject's foot via an intermediate portion of the sole separating the vibration actuator 108 and the subject's foot.

In one example the system can be used in a movement assistance program to generate appropriate alert signals to reduce the likelihood that elderly or otherwise vulnerable subjects fall over.

During the calibration phase, the subject walks around wearing the shoes and corresponding calibration sensor data is generated by the sensors. This calibration data is then sent to the remote computing system 112.

The gait characterising function 113 uses this calibration sensor data to generate gait parameter data relating to the timing of the subject's gait swing when walking (that is, the period of time taken for the subject to complete a stride).

A subject's swing time can typically be determined reliably with simple algorithms, and its value can be continuously updated. The swing time parameter can be generated by the gait characterising function 113 identifying from the sensor data the time delay between “toe-off” and “heel-strike” events for each foot.

Specifically, from the calibration sensor data, the gait characterising function 113 is configured to detect toe-off and heel-strike events by applying a peak-detection algorithm to sensor data associated with the ankle's angular movement rate.

Sensor data associated with a step typically features two peaks, each in proximity of toe-off and heel-strike. Combining this information with sensor data associated with the vertical acceleration (lift-off at toe-off and impact at heel-strike) enables real-time estimates of toe-off and heel-strike events to be generated.

The gait characterising function 113 is configured to identify from the calibration sensor data a number of toe-off and heel-strike events and generate a plurality of swing time values.

The gait characterising function 113 is then configured to generate, from this plurality of swing time values, an average swing time value which is an average of the plurality of swing time values.

The gait characterising function 113 is further configured to calculate, using the plurality of swing time values, a time value corresponding to one standard deviation from this average swing time value of the plurality of swing time values.

The gait characterising function 113 is configured to communicate swing time gait parameter data to the sensor value prediction function 114 comprising the average swing time value and the time value corresponding to one standard deviation calculated from the plurality of swing time values).

The sensor data value prediction function 114 is configured to process this swing time gait parameter data in accordance with parameters specified by the movement assistance program stored in the program parameter database 117, to generate a distribution of sensor data values that are predicted to arise in a way suggesting an imminent fall.

In this example, the program parameters specify a number of standard deviations from a subject's average swing time by which the subject's swing time will increase if a fall is predicted to be imminent.

The sensor data value prediction function 114 processes the average swing time gait parameter and the standard deviation time parameter in accordance with the standard deviations specified in the program parameter database 117 to generate a distribution of sensor data values that are predicted to arise if a fall is imminent. These sensor values will correspond, for example, with the distribution of sensor data values that will arise if the toe-off and heel-strike events sequentially occur with an above-threshold time separation.

For example, the gait characterising function 113 may calculate from the calibration sensor data that the subject's average swing time is 700 ms with a distribution of swing times such that one standard deviation from the average swing time is 250 ms. The gait parameter data communicated from the gait characterising function 113 to the sensor data value prediction function 114 will therefore specify an average swing time value of 700 ms and a standard deviation time value corresponding to one standard deviation of 250 ms.

Further, the program parameter database 117 may comprise program parameter data specifying that if a threshold increase in a subject's swing time of two deviations from the average swing time is met or exceeded, this is indicative of an imminent fall.

Thus in this example, this will be a gait swing time of at least:

700 ms+(2×250 ms)=1200 ms

In such an example, the sensor data value prediction function 114 is configured to generate a distribution of sensor data values that are predicted to arise if the subject's swing time is 1200 ms or greater.

Sensor value distribution data comprising this distribution of sensor data values is then communicated back to the sensory stimulation unit in each shoe.

During an operational phase, the subject then moves around wearing the shoes. In the event that the subject's gait swing time changes to exceed 1200 ms, the sensor data monitoring function 115 identifies that the sensor data generated by the sensor unit exceeds the threshold gait swing time value and generates an imminent fall gait event detected signal.

This imminent fall gait event detected signal is communicated to the motor control function 116. The motor control function 116 generates a corresponding control signal which when received by the vibration actuator 108, causes the vibration actuator 108 to generate a corresponding sensory stimulation to alert the subject that they might be about to fall.

The subject, thus alerted, may then be less likely to fall.

The detection of sensor data associated with swing time by the sensor data monitoring function 115 typically occurs at a rate higher than customary human reaction times. In this way, the subject experiences smooth and “instantaneous” feedback, as soon as the set threshold is passed, and vibration is triggered. Typically, human reaction times are less than 0.1 s, detecting the subject's gait swing time at 100 Hz will result in smooth operation.

In another example, the system can be used in a program of therapy to assist subjects suffering from neurological disorders such as multiple sclerosis or Parkinson's disease and who experience “gait freeze”.

In such an example, in keeping with the previous example, during a calibration phase, the subject walks around wearing the shoes and corresponding calibration sensor data is sent to the remote computing system 112. The gait characterising function 113 uses this calibration sensor data to generate gait parameter data including a swing time gait parameter associated with the subject's gait swing time when walking normally.

The program parameter database 117 has stored therein program parameters specified by a therapy program which specify a reduction in average swing time of more than a predetermined amount (e.g. 50%) over a predetermined period (e.g. 60 seconds) is indicative of an impending gait freeze.

The sensor data value prediction function 114 processes the swing time gait parameters identified by the gait characterising function 113, in accordance with parameters specified by a therapy program stored in the program parameter database 117, to generate a predicted distribution of sensor data values that are predicted to arise in the event that the subject's average swing time decreases by the predetermined amount over the predetermined period of time.

Corresponding sensor value distribution data is communicated back to the sensory stimulation unit in each shoe.

During an operational phase, the subject then moves around wearing the shoes.

In the event that the subject's average swing time reduces by at least the threshold amount (e.g. at least 50%) during the threshold period of time (e.g. within 60 seconds) indicating an impending episode of gait freeze, the sensor data monitoring function 115 identifies that the sensor data generated by the sensor unit falls within the predicted distribution of sensor data values associated with an average swing time decrease above the threshold value and generates an “gait freeze” imminent gait event detected signal.

This “gait freeze” imminent gait event detected signal is communicated to the motor control function 116. The motor control function 116 generates a corresponding control signal which when received by the vibration actuator 108, causes the vibration actuator 108 to generate a corresponding sensory stimulation to alert the subject that initiate a step and end the gait freeze. The subject, thus alerted, may then be less likely to suffer from an episode of gait freeze.

In another example, the system can be used in a program of training to assist a subject seeking to improve their technique when performing an activity such as running.

In such an example, during a calibration phase, the subject moves around wearing the shoes, and in particular moves around with a particular desired movement form. Corresponding calibration sensor data is sent to the remote computing system 112.

The gait characterising function 113 uses this calibration sensor data to generate gait parameter data indicative of one or more gait parameters associated with the desired movement form, for example step width and swing time.

The program parameter database 117 has stored therein program parameters specified by a training program which specify that for effective training, the subject must attempt to avoid deviations from these gait parameters by more than a predetermined amount, for example 5%.

The sensor data value prediction function 114 processes the swing time gait parameters identified by the gait characterising function 113, in accordance with parameters specified by a movement training program and stored in the program parameter database 117, to identify a predicted distribution of sensor data values corresponding to a variation in the gait parameters that are predicted to arise if the subject deviated from the desired movement form by the specified amount (e.g. variations in swing time and step width of more than 5%).

Corresponding sensor value distribution data is communicated back to the sensory stimulation unit in each shoe.

During an operational phase, the subject then moves around wearing the shoes and in particular attempting to maintain the desired movement form.

In the event that the subject's movement deviates from the desired form by the amount specified by the training program parameters, the sensor data monitoring function 115 identifies that the sensor data generated by the sensor unit falls within the predicted sensor data associated with this deviation and generates a form deviation gait event detected signal.

This form deviation gait event detected signal is communicated to the motor control function 116. The motor control function 116 generates a corresponding control signal which when received by the vibration actuator 108, causes the vibration actuator 108 to generate a corresponding sensory stimulation to alert the subject that they have deviated from the desired form.

Typically, responsive to receiving a gait event detected signal, the motor control function 116 generates a control signal which when received by the vibration actuator 108 causes the vibration actuator 108 to generate stimulating vibration which is transferred to the subject's foot.

However, in certain examples, the gait event detected signal causes the motor control function 116 to generate a control signal which when received by the vibration actuator 108 causes the vibration actuator 108 to cease the application of vibratory stimulation. For example, the data processor 107a and motor control function 116 may separately be configured, in accordance with a program of training to provide a regular sequence of confirmatory vibrations which convey to a subject (such as an athlete) that they are moving at a desired pace. In such an example, the predicted distribution of sensor data values may be indicative of sensor values which will be generated in the event that the subject drops below or exceeds the desired pace. Upon detection that the subject has dropped below or exceeded this pace by the sensor data falling within this distribution of sensor data values, a corresponding pace discrepancy gait event detected signal is generated by the sensor data monitoring function 115 which is communicated to the motor control function 116 which then ceases the generation of the confirmatory vibrations until the desired pace is achieved again.

The program parameters stored in the program parameter database 117 are defined based on the type of program being provided (e.g. a training program, a therapy program, or a program of movement assistance). In each case the program parameters may be selected based on relevant research. For example, the program parameters for a therapy program attempting to alleviate or reduce the occurrence of gait freeze will be defined in part based on what research into this condition suggests is effective for treating the condition.

The program parameters may also be defined in part by characteristics of the subject who will be using the system. For example, characteristics such as age, weight, sex, height and so on. For example, in programs for providing movement assistance which aim to reduce the likelihood of subjects falling, the variation in swing time that is identified to predict an imminent fall may vary in dependence on the age of the subject.

The program parameters may also be defined in part by historic data associated with the subject. For example, a particular subject may have previously exhibited a certain sequence of gait kinematics ahead of the occurrence of an episode of gait freeze. in such examples, the program parameters may be selected to specify these gait kinematics.

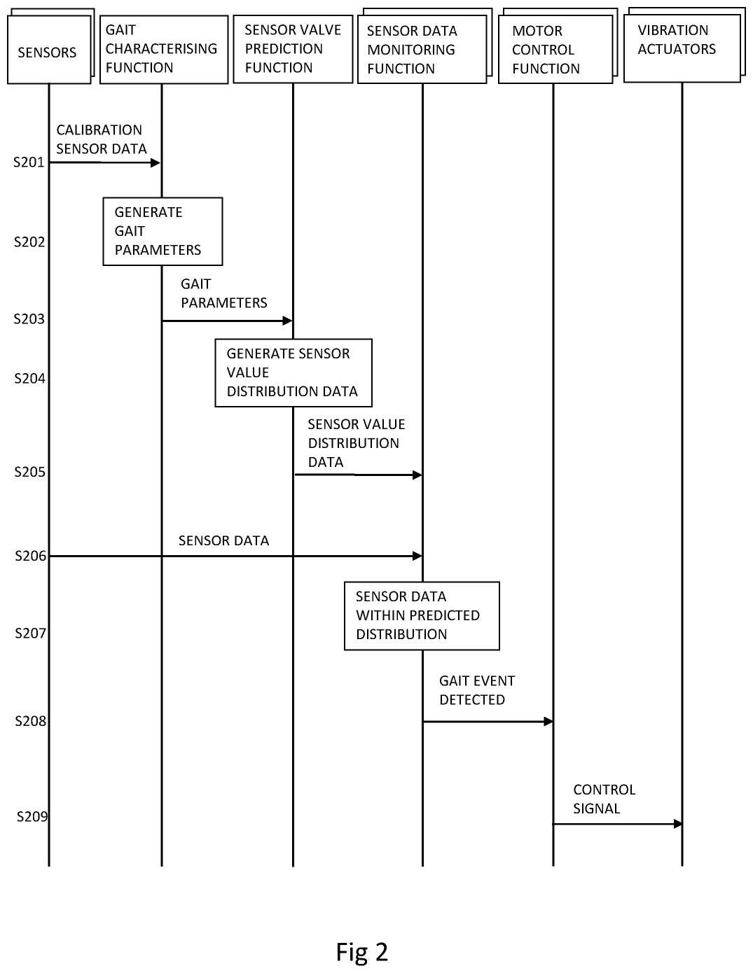

FIG. 2 provides a schematic diagram summarising the operation of the system as described above.

At a first stage S201 calibration sensor data is communicated from the sensors from the sensory stimulation unit of both shoes of the pair of shoes to the remote computing system to be processed by the gait characterising function.

At a second stage S202 the gait characterising function processes the calibration sensor data to generate gait parameter data associated with one or more gait parameters associated with the subject's gait kinematics.

At a third stage S203 the gait characterising function communicates the gait parameter data to the sensor data value prediction function.

At a fourth stage S204, the sensor data value prediction function, using the gait parameter data and program parameter data from the program parameter database, generates a predicted distribution of sensor data values within which the sensor data generated by the sensor units of each shoe would fall in the event that a subject's gait kinematics change in a way requiring stimulating vibration to be applied in accordance with parameters specified by a program of therapy, program of movement assistance or program of training.

At a fifth stage S205 the sensor value distribution data is communicated to the sensor data monitoring function running on the data processor of each shoe.

At a sixth stage S206, at each shoe, sensor data from the sensor unit is received by the sensor data monitoring function.

At a seventh stage S207, at each shoe, the sensor data is monitored to determine if it falls within the predicted distribution of sensor data values.

At an eighth stage S208, in the event that the sensor data monitoring function determines that the sensor data falls within the predicted distribution of sensor data values, a gait event detected signal is communicated to the motor control function.

At a ninth stage S209, at each shoe, responsive to receipt of the gait event detected signal, the motor control function generates a control signal which is communicated to the vibration actuators responsive to which vibration for sensory stimulation of the subject's foot is generated.

In certain examples, the first stage S201, second stage S202, third stage S203, fourth stage S204 and fifth stage S205 are periodically repeated. In other words, periodically, updated calibration sensor data is sent from the sensory stimulation unit 104 to the remote computing system 112 enabling the gait characterising function 113 to periodically recharacterize the subject's gait by generating updated gait parameters which are then used by the sensor data value prediction function 114 to generate updated sensor value distribution data which is then communicated back to the sensor data monitoring function 115 running on the data processor 107a of each shoe 101a, 101b. This update process may occur at any suitable frequency, for example daily or hourly. In certain examples, the update process can be manually triggered, for example based on a suitable command signal communicated to the sensory stimulation unit from the remote computing system 112.

Advantageously, this means that the training program, movement assistance program or therapy program can continue to be effectively adapted as the subject's gait kinematics change over time, for example, due to changes brought about by the training program or therapy program.

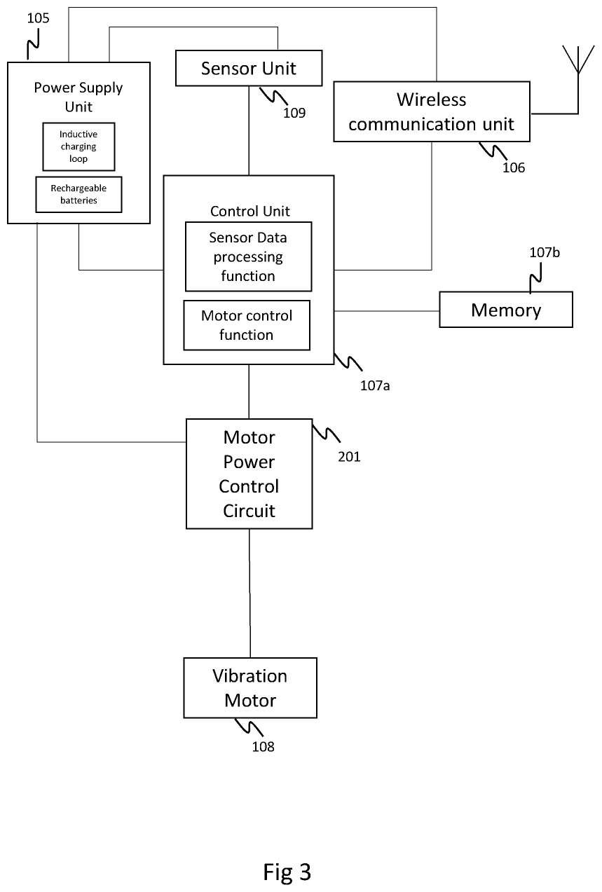

FIG. 3 provides a schematic diagram providing a more detailed view of a sensory stimulation unit 104 arranged in accordance with certain embodiments of the invention.

As can be seen, the sensory stimulation unit 104 comprises a power supply unit 105 which, in certain embodiments comprises one or more rechargeable batteries 105a and an inductive charging loop 105b for charging the rechargeable batteries 105a via a wireless charging unit.

The sensory stimulation unit 104 further comprises the data processor 107a which can be provided by any suitable programmable microprocessor or by other appropriate data processing means, for example a custom-designed integrated circuit such as a field programmable gate array (FPGA).

The data processor 107a is connected to a motor power control circuit 201 via a suitable signal line which is connected, via a further suitable signal line, to the vibration actuator 108.

The vibration actuator is typically provided by an electric motor comprising an eccentrically mounted weight on a shaft of the motor. However, the vibration actuator can be provided by other suitable electro-mechanical devices, for example piezoelectric vibration actuators and voice-coil-like linear electromagnetic actuators (“tactors”).

The sensor unit 109 is connected via a suitable signal line to the data processor 107a. The sensor unit 109 is typically provided by an inertial measurement unit (IMU) comprising an accelerometer, gyroscope, and magnetometer, connected to the data processor unit.

The wireless communication unit 106 is also connected to the data processor 107a via a suitable signal line. The wireless communication unit 106 can be provided by any suitable wireless communication unit operating in accordance with conventional radio protocols such as Bluetooth, Zigbee, LoRa, NFC, WiFi and so on. In certain examples the wireless communication unit 106 can be provided by a data transceiver provided with a subscriber identity module (SIM) and enabling data to be transmitted to and from the data network 110 via a cellular mobile telephone network.

All of the components of the sensory stimulation unit 104 are connected to the power supply unit 105 via suitable power lines.

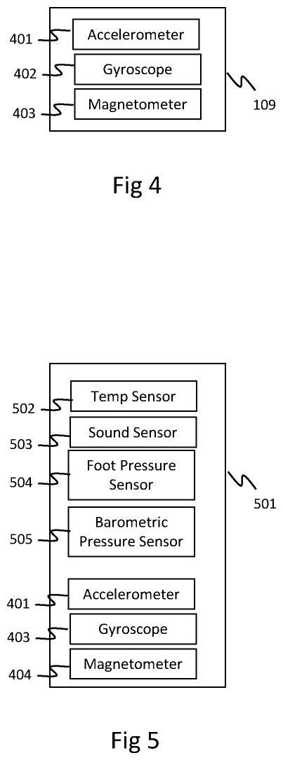

FIG. 4 provides a schematic diagram depicting in more detail the sensors of the sensor unit 109 in accordance with certain embodiments of the invention. As can be seen from FIG. 4, the sensor unit 109 comprises an accelerometer 401, a gyroscope 402 and a magnetometer 403. This combination of sensors typically provides enough information about a subject's movement to enable aspects of the subject's gait to be characterised including gait speed, step velocity, step length, swing time variability, stride length, step width, rhythm (such as step time, swing time, stance time, single support, double support), variability (such as step velocity variability, step length variability, step time variability, stance time variability), asymmetry (such as swing time asymmetry, step time asymmetry, stance time asymmetry), postural control (such as step length asymmetry), step characteristics (strike angle, minimum toe clearance, foot angles (such as supination angle, strike angle, lift-off angle, angular velocity) and peak parameters such as peak propulsion and peak braking.

In certain embodiments, the sensor unit 109 may comprise one or more further sensors.

FIG. 5 provides a schematic diagram depicting a further example of a sensor unit 501 comprising the sensors of the sensor unit 109 shown in FIG. 4 comprising further sensors, specifically, a temperature sensor 502, a sound sensor 503, a foot pressure sensor 504, and a barometric pressure sensor 505.

In use, the temperature sensor 502 is configured to measure the temperature and generate corresponding temperature data. This temperature data is communicated to the data processor 107a. The data processor 107a is configured to use this temperature data to calibrate, if needed, sensor data from the sensor unit 109 to account for changes (drift) in the output of the sensor unit 109 arising due to changes in temperature to which the system is exposed.

In certain examples, the data processor 107a is configured to control the wireless communication unit 106 to communicate the sensor data generated by the sound sensor 503, the foot pressure sensor 504, and the barometric pressure sensor 505 to the remote computing system of further functioning.

In use, the foot-pressure sensor 504 is typically positioned so that pressure changes arising due to the subject contacting the ground can be detected. For example, the foot-pressure sensor 504 can be provided by a two-dimensional pressure sensing pad configured to be positioned across the base, or part of the base, of a modified shoe sole so that the pressure at different contact points of the subject's foot as it contacts the ground can be measured. The foot-pressure sensor 504 is configured to generate pressure data. This pressure data is communicated to the remote computing system 112. The gait characterising function 113 is configured to use the pressure data when generating the gait parameters. For example, the gait characterising function 113 can use the pressure data to determine points in time when the subject's foot is in contact with the ground and/or using it to determine points in time when particular regions of the subject's foot (for example the ball and the heel) are in contact with the ground. Further information relevant to analysing a subject's gait can be determined from the pressure data such as the impact force with which a subject is contacting the ground with their foot or specific regions of their foot.

In use, the sound sensor 503 is configured to detect sound in the region of the subject's foot and to generate corresponding sound data. This sound data is communicated to the remote computing system 112. In certain embodiments, the gait characterising function is configured to use the sound data to classify the type of surface that the subject is moving (walking, running) on which can then be used to refine the algorithms used to estimate the subject's gait parameters. In certain examples, the sound data can be used to detect the type or place of the subject's activity.

In use, the barometric pressure sensor 505 is configured to detect the atmospheric pressure around the shoe and generate corresponding barometric pressure sensor data. This barometric pressure sensor data is communicated back to the remote computing system which in certain embodiments has running thereon an altitude detection function which is configured to receive the sensor data from the barometric pressure sensor to generate corresponding altitude data. This altitude data can be used to track vertical movement of the subject whilst wearing the shoes, for example as part of an exercise program.

In certain embodiments the altitude detection function can be incorporated in a movement distance analysis function as described in more detail below.

In the example described with reference to FIG. 1a, the remote computing system is provided by one or more application servers and data is communicated between the remote device and the sensory stimulation unit via the data network. The program parameters are stored in a program parameter database which is connected to the remote computing system. The skilled person will understand that the arrangement of these components is one example of how a system in accordance with embodiments of the invention can be arranged and the components of the system can be manifested in any suitable alternative way.

For example, in other configurations the remote computing system can be provided by a personal computing device such as a personal computer (“PC”), tablet computer, smartphone or similar and the program parameters stored on such a device in a suitable memory.

In certain embodiments, for example where the remote computing system is provided by a personal computing device, each sensory stimulation unit and the remote computing system may communicate directly via a suitable data link, that is, without an intermediate data network and/or without an intermediate base station as is shown in FIG. 1a. For example, the remote computing system and each sensory stimulation unit may communicate data with each other via a short-range radio protocol such as Bluetooth, WiFi or similar.

In embodiments of the invention, the sensor stimulation unit can be configured (by virtue of the positioning of the vibration actuator with respect to the sole of the item of footwear) to stimulate any suitable region on the underside (inferior) side of a subject's foot (the sole of the foot).

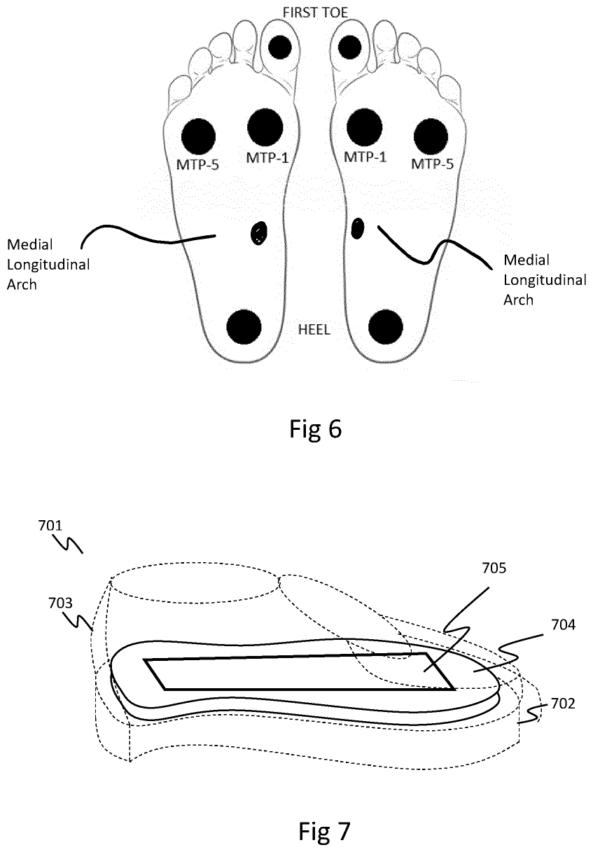

These regions include the first metatarsophalangeal joint; the fifth metatarsophalangeal joint, region of the heel; the region of the big toe and the medial longitudinal arch. These example regions are shown in FIG. 6. In examples in which the item of footwear incorporates a single vibration actuator, that actuator may be positioned at any one of the positions, although the skilled person will recognise that other locations not shown in FIG. 6 are possible.

In certain examples, each item of footwear may be provided with more than one vibration actuator. In such examples, the sensory stimulation unit can be configured such that the vibration actuators are positioned to provide sensory stimulation to any suitable combination of regions of the underside of the subject's foot. For example, a first vibration actuator can be positioned so as to stimulate a foot position in the region of the first metatarsophalangeal joint; a second vibration actuator can be positioned so as to stimulate a foot position in the region of the fifth metatarsophalangeal joint, and a third vibration actuator can be positioned in the so as to stimulate a foot position in the region of the heel. In certain other embodiments, a fourth vibration actuator can be positioned so as to stimulate a foot position in the region of the big toe.

In certain embodiments the number of vibration actuators and the position of vibration actuators will be selected based on the type of therapy or training being delivered to the subject because, for example, stimulation in different locations can induce different reactions in different patient groups.

In the examples above, the foot stimulation vibration can be “sensory” so that the subject is consciously aware of the vibration. This is an example of “tactile cuing” whereby the subject receives “cues” via consciously detectable tactile stimulation.

However, in certain examples, for example where vibration is being applied as therapy for example for diabetic neuropathy patients, or to prevent falls or in response to foot freeze, the vibration can be sub-sensory such that the subject may not be consciously aware of the vibration, but the vibration nevertheless generates neurological stimulation and produces a desired effect such as improving balance and walking. In such examples, the foot stimulating vibration generated by the vibration actuator or vibration actuators is subsensory vibration (not consciously detectable by the subject).

Particularly in examples in which subsensory vibrations are generated, the data processor associated with each item of footwear can be configured to calibrate the vibration generated by the vibration actuator (or each vibration actuator) to take account of the fact that different subjects have different sensory threshold levels, and these sensory thresholds will vary across different areas of a subject's foot.

To facilitate this, the data processor associated with each item of footwear can be configured to implement a calibration process which controls the vibration actuator (or each vibration actuator) to iteratively step through a sequence of different vibration levels until a vibration level is identified, that is just below a subject's sensory perception for the region of the subject's foot that the vibration actuator stimulates. This calibration process can be carried out in conjunction with an external device, for example a mobile computing device, such as a smart phone, connected to the data processor via the data transceiver and a suitable wireless link.

Different vibration levels can be provided by the vibration actuators vibrating at different frequencies (for example when the vibration actuators are provided by electric motors comprising an eccentrically mounted weight on a shaft of the motor) and/or the vibration actuators vibrating at different amplitudes (for example where the vibration actuators are provided by voice-coil-like linear electromagnetic actuators (“tactors”)).

In this way, after the calibration process is completed, a vibration level (typically consisting of a vibration frequency and/or vibration amplitude) will be determined for each vibration actuator which is then used during operation of the system.

In certain examples, the data processor in each item of footwear controls the vibration actuator (or each vibration actuator) to generate the foot stimulating vibration using “stochastic resonance”. In such examples, the foot stimulating vibration is generated in accordance with a random pattern (which is typically more effective for neuro-stimulation). For example, the vibration actuators can be configured to apply the foot stimulating vibration in an “on/off” pattern, with the time between the “on” and “off” phases varying randomly between, for example, 0.01 s and 0.09 s.

In certain examples, sensory stimulating apparatus in accordance with embodiments of the invention can be incorporated in a modified insole which can be inserted and removed from an item of footwear. An example of such an embodiment is depicted in FIG. 7. FIG. 7 provides a simplified schematic diagram showing an otherwise conventional item of footwear 701 comprising a sole 702 and an upper 703 (the sole 702 and upper 703 are indicated with broken lines and shown transparently). A removable modified insole 704 is shown incorporated within which is an assembly 705 comprising a vibration generating apparatus.

As will be understood, the removable modified insole 704 can be removed from the item of footwear 701 and placed in a different item of footwear. This allows, for example, the modified insole 704 to be used in the footwear of multiple subjects or multiple items of footwear of the same subject. The modified insole 704 may comprise a washable and/or otherwise disinfectable outer which enables the modified insole 704 to be cleaned, for example, for the purposes of hygiene, after use in the footwear of a first subject and before use in the footwear of a second subject.

In the example described with reference to FIG. 1a, all the components associated with detecting the motion of the subject and applying the sensory stimulation are incorporated in a single sensory stimulation unit. However, in other examples these components maybe integrated with the item in footwear in different ways. For example, the vibration actuator or vibration actuators can be fitted to a modified shoe sole or modified insole whilst other components, for example the sensors and the data processor, can be incorporated in other parts of the item of footwear, such as, for example, a shoe upper or a shoe tongue.

Embodiments of the invention can be used with any suitable form of footwear. Such footwear includes shoes such as trainers (sneakers), boots and sandals. In certain embodiments, vibration generating apparatus can be incorporated in specific medical footwear such as controlled ankle motion (CAM) walking boots (“moonboots”).

In certain embodiments the sensory stimulation unit of one or both shoes are configured to implement a movement distance tracking function. The movement distance tracking function is configured to track the distance the shoe has moved and generate corresponding movement distance data. This movement distance data can then be communicated back to the remote computing system by the data transceiver for analysis by a movement distance analysis function.

The movement distance analysis function may be configured to analyse the movement distance data to determine movement patterns associated with movement of the shoe (for example, total distance moved, average time moving, maximum and minimum movement distances over set periods and so on) and generate corresponding movement distance analysis data. The movement distance analysis data can then be used to optimise the program of therapy, movement assistance or training being provided by the system. For example, a specialist (such as a physician) can manually change the program parameters stored in the program parameter database based on a pattern of movement distance by the subject.

The movement distance tracking function can be implemented by any suitable means. For example a movement distance tracking function can be implemented on the data processor on the sensory stimulation unit of one or both shoes and the sensory stimulation unit can be further equipped with a location tracking device (such as a global navigation satellite system (GNSS) receiver, for example a GPS receiver). The data processor is configured to receive location data from the location tracking device and from this generate movement distance data which is communicated back to the remote computing system. In other examples, the movement tracking function can be configured to use the sensor data collected by the sensor unit (for example, inferring a total distance moved by estimating the number of steps taken by the subject) and from this generate movement distance data which is communicated back to the remote computing system.

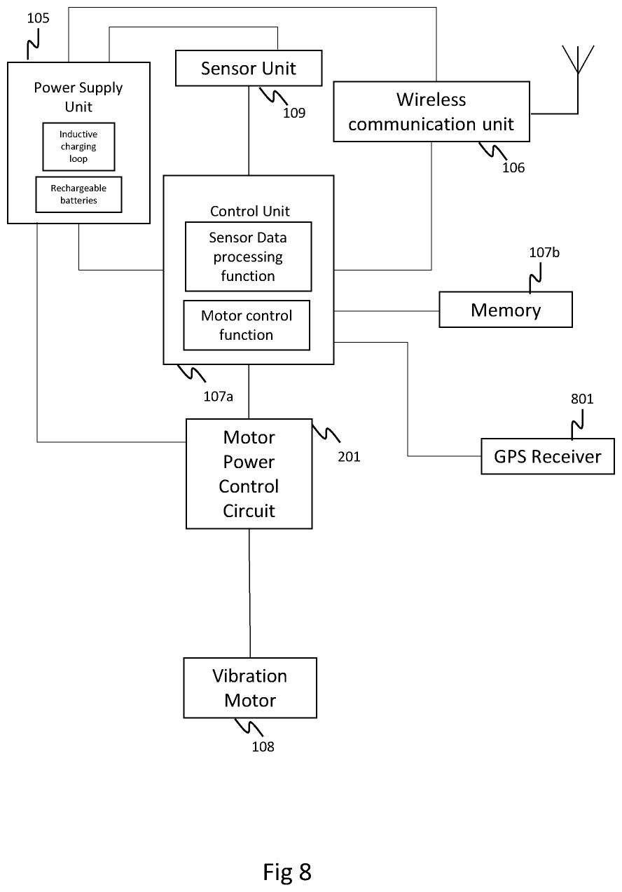

FIG. 8 provides a schematic diagram of a sensory stimulation unit arranged for this purpose. FIG. 8 shows a sensory stimulation unit corresponding to the sensory stimulation unit described with reference to FIG. 3, except that it further comprises a location tracking device 801 provided by a GNSS receiver such as a GPS receiver.

As described above, in certain embodiments the movement distance analysis function can incorporate the altitude detection function so that movement patterns associated with altitude (for example, number of metres ascended and/or descended during a given period of time) can also be taken into account when generating movement distance analysis data.

In certain embodiments, the system is provided with further functionality that enables sensory stimulation to be generated for further purposes.

For example, in certain embodiments, the system is configured to provide tactile cueing to prompt the subject during training or testing.

Such prompting can include prompting the subject to perform actions such as start, stop, turn, sit down, stand up and so on.

Such embodiments can be implemented in any suitable way. In one example, the remote computing system is provided with a tactile cueing control function which communicates tactile cueing data to the sensor unit on one or both shoes.

The data processor of the sensor unit has running thereon a tactile cueing generation function which responsive to the tactile cueing data received from the tactile cuing control function running on the remote computing system is configured to generate suitable control signals for controlling the vibration actuator to generate tactile cueing vibrations.

In this way, for example, a sequence of tactile cueing vibrations could be generated prompting the subject to start walking then stop walking then start walking again and so on during a training or therapy session.

In the example embodiments described above, the vibration actuators of the sensory stimulation unit are positioned and configured such that sensory stimulation is applied principally to the underside (inferior side) of a subject's foot, that is the sole of a subject's foot.

However, in other embodiments, the sensory stimulation unit may be configured alternatively or additionally to apply sensory stimulation to other regions of subject's foot. For example, in certain embodiments, an item of footwear is provided incorporating a sensory stimulation unit substantially corresponding to those described above except that the vibration actuator or vibration actuators of the sensory stimulation unit are positioned and configured to apply sensory stimulation to the subject's ankle or region immediately adjacent to the subject ankle.

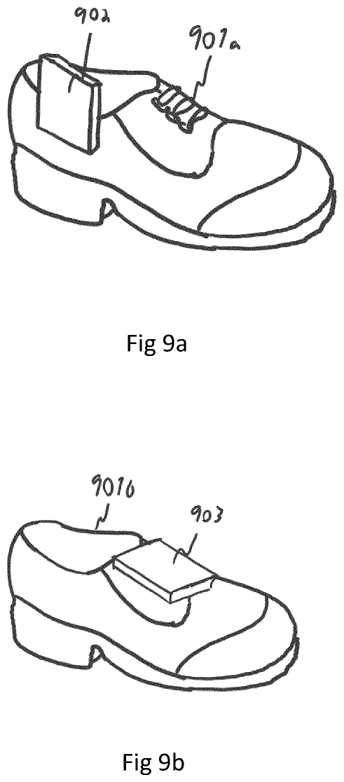

FIG. 9a provides a simplified schematic diagram of such an embodiment. FIG. 9a shows an item of footwear 901a comprising a sensory stimulation unit 902 of the type described above and including, for example, all the components depicted in FIG. 3. As can be seen from FIG. 9a, the sensory stimulation unit 902 is mounted to the item of footwear 901 a in a position such that during use sensory stimulation will be applied to the subject's distal ankle.

In further embodiments, an item of footwear is provided incorporating a sensory stimulation unit substantially corresponding to those described above except that the vibration actuator or vibration actuators of the sensory stimulation unit are positioned and configured to apply sensory stimulation to an upper (superior) side of the subject's foot.

FIG. 9b provides a simplified schematic diagram of such an embodiment. FIG. 9b shows an item of footwear 901b comprising a sensory stimulation unit 903 of the type described above and including, for example, all the components depicted in FIG. 3. As can be seen from FIG. 9b, the sensory stimulation unit 902 is mounted to the item of footwear 901a in a position such that during use, sensory stimulation will be applied to the upper (superior) side of the subject's foot.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent, or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A system for applying sensory stimulation to a subject's foot or ankle based on detected gait kinematics for therapy, training, or movement assistance, said system comprising:
at least one item of footwear incorporating one or more sensors, one or more vibration actuators, a data processor, memory, and a data transceiver, and
a remote computing system comprising data communication means and data processing means, wherein
said sensors are configured, during a calibration phase, to generate calibration sensor data associated with movement of a subject wearing the item of footwear and said data transceiver is configured to communicate the calibration sensor data to the remote computing system, and
said remote computing system is configured to receive, via the data communication means, the calibration sensor data from the item of footwear, and
process, using the data processing means, the calibration sensor data to:
generate one or more gait parameters associated with the subject's gait kinematics;
generate, using the gait parameters and one or more program parameters associated with a program of therapy, training, or movement assistance, a predicted distribution of sensor data values within which further sensor data generated by the one or more sensors is predicted to fall in the event of movement of the subject requiring sensory stimulation to be applied in accordance with the program of therapy, training or movement assistance, said remote computing system configured to communicate, via the data communication means, the predicted distribution of sensor data values to the data transceiver, and
said data transceiver is configured to receive the predicted distribution of sensor data values and said memory is configured to store the predicted distribution of sensor data values, whereupon, during an operational phase
said data processor is configured to monitor further sensor data from the one or more sensors, and, in the event that the further sensor data from the one or more sensor data falls within the predicted distribution of sensor data values, said data processor is configured to control the one or more vibration actuators to provide sensory stimulation in accordance with the program of therapy, training or movement assistance.

2. A system according to claim 1, wherein, during a further calibration phase, the sensors are configured to generate further calibration sensor data associated with subsequent movement of the subject and said data transceiver is configured to communicate the further calibration sensor data to the remote computing system, said remote computing system is configured to receive, via the data communication means, the further calibration sensor data, process, using the data processing means, the further calibration sensor data to:
generate one or more updated gait parameters associated with the subject's gait kinematics;
generate, using the updated gait parameters and one or more program parameters associated with a program of therapy, training, or movement assistance, an updated predicted distribution of sensor data values within which the further sensor data generated by the one or more sensors is predicted to fall in the event of movement of the subject requiring sensory stimulation to be applied in accordance with the program of therapy, training or movement assistance, said remote computing system configured to communicate, via the data communication means, the updated predicted distribution of sensor data values to the data transceiver, and
said data transceiver is configured to receive the updated predicted distribution of sensor data values and said memory is configured to store the updated predicted distribution of sensor data values, whereupon, during a further operational phase
said data processor is configured to monitor the further sensor data from the one or more sensors, and, in the event that the sensor data from the one or more sensor data falls within the updated predicted distribution of sensor data values, said data processor is configured to control the one or more vibration actuators to provide sensory stimulation in accordance with the program of therapy, training or movement assistance.

3. A system according to claim 2, wherein the data processing means is configured to periodically generate the updated predicted distribution of sensor data values at a predetermined interval and/or responsive to an update signal from the remote computing system

4. A system according to claim 1, wherein the gait parameters include one or more of gait speed, step velocity, step length, swing time variability, stride length, step width, rhythm, variability, asymmetry, postural control and step characteristics.

5. A system according to claim 1, wherein the one or more vibration actuators are configured to generate sub-sensory vibration.

6. A system according to claim 1, wherein the item of footwear incorporates a plurality of vibration actuators.

7. A system according to claim 1, wherein the data processor is configured to actuate each vibration actuator to generate foot stimulating vibration at a vibration level determined during a calibration process in which a subject's sensory perception is assessed at each foot position corresponding to a position of each vibration actuator thereby accommodating differences in sensory perception across the subject's foot.

8. A system according to claim 7, wherein the vibration level comprises a predetermined vibration frequency and/or predetermined vibration amplitude.

9. A system according to claim 1, wherein the sensory stimulation is tactile cueing.

10. A system according to claim 1, whereby the one or more vibration actuators are embedded in a sole or insole of the at least one item of footwear.

11. (canceled)

12. (canceled)

13. A system according to claim 1, wherein the sensors comprise one or more inertial measurement units comprising one or more of an accelerometer, gyroscope, and magnetometer.

14. A system according to claim 13, wherein the sensors further comprise one or more of a foot-pressure sensor for detecting pressure changes arising due to the subject contacting the ground, a temperature sensor for detecting an ambient temperature, a barometric pressure senor for detecting barometric pressure and a sound sensor.

15. A system according to claim 1, wherein the at least one item of footwear further incorporates movement distance tracking means configured to generate movement distance data associated with a distance moved by the item of footwear, and
said remote computing system has running thereon a movement distance analysis function, wherein
said data transceiver is configured to communicate the movement distance data to the remote computing system for analysis by the movement distance analysis function to generate movement distance analysis data.

16. A system according to claim 1, wherein the program of therapy, training or movement assistance is a program of movement assistance for alerting a subject to a potential fall, said predicted distribution of sensor data values corresponding to a range of sensor values predicted to arise in the event the subject's gait kinematics change in such a way indicating an imminent fall, said data processor thereby operable to control the one or more vibration actuators to generate sensory stimulation in the event of an imminent fall being detected.

17. A system according to claim 1, wherein the program of therapy, training or movement assistance is a program of therapy for alerting a subject to a potential episode of gait freeze, said predicted distribution of sensor data values corresponding to a range of sensor values predicted to arise in the event the subject's gait kinematics change in such a way indicating an imminent episode of gait freeze, said data processor thereby operable to control the one or more vibration actuators to generate sensory stimulation in the event of an imminent episode of gait freeze detected.

18. A system according to claim 1, wherein the program of therapy, training or movement assistance is a program of training for alerting a subject to movement that deviates from a desired movement form, said predicted distribution of sensor data values corresponding to a range of sensor values predicted to arise in the event the subject's gait kinematics change in such a way indicating that the desired movement form has been deviated from, said data processor thereby operable to control the one or more vibration actuators to generate sensory stimulation in the event of a desired movement form has been deviated from.

19. A system according to claim 1, wherein the program of therapy, training or movement assistance is a program of training for alerting a subject to movement that is in accordance with a desired movement form, said predicted distribution of sensor data values corresponding to movement of the subject in the event the subject's gait kinematics indicate that a desired movement form is being maintained, said one or more vibration actuators thereby providing sensory stimulation in the event of a desired movement form is maintained.

20. (canceled)

21. (canceled)

22. (canceled)

23. A method of applying sensory stimulation to a subject's foot or ankle based on detected gait kinematics for therapy, training, or movement assistance, said method comprising, during a calibration phase:
generating at an item of footwear calibration sensor data associated with movement of a subject wearing an item of footwear;
communicating the calibration sensor data from the item of footwear to a remote computing system,
generating, at the remote computing system, using the calibration sensor data, one or more gait parameters associated with the subject's gait kinematics;
generating, at the remote computing system, using the gait parameters and one or more program parameters associated with a program of therapy, training, or movement assistance, a predicted distribution of sensor data values within which further sensor data generated at the item of footwear is predicted to fall in the event of movement of the subject requiring sensory stimulation to be applied in accordance with the program of therapy, training or movement assistance;
communicating the predicted distribution of sensor data values to the item of footwear, and, during an operational phase: monitoring further sensor data generated at the item of footwear, and, in the event that the further sensor data falls within the predicted distribution of sensor data values, controlling one or more vibration actuators to provide sensory stimulation in accordance with the program of therapy, training or movement assistance.

24. An arrangement for fitting to an item of footwear for use in a system according to claim 1, said arrangement comprising one or more sensors, one or more vibration actuators, a data processor, memory, and a data transceiver, wherein
said sensors are configured, during a calibration phase, to generate calibration sensor data associated with movement of a subject wearing the item of footwear and said data transceiver is configured to communicate the calibration sensor data to a remote computing system, and
said data transceiver is configured to receive a predicted distribution of sensor data values from the remote computing system and said memory is configured to store the predicted distribution of sensor data values, whereupon, during an operational phase
said data processor is configured to monitor further sensor data from the one or more sensors, and, in the event that the further sensor data from the one or more sensor data falls within the predicted distribution of sensor data values, said data processor is configured to control the one or more vibration actuators to provide sensory stimulation in accordance with the program of therapy, training or movement assistance.

25. (canceled)

26. (canceled)

27. A computer program for running on a data processor incorporated in an item of footwear and for use in a system according to claim 1, said computer program comprising instructions which when implemented on a data processor, controls the data processor to perform a method comprising:
generating at the item of footwear calibration sensor data associated with movement of a subject wearing an item of footwear;
communicating the calibration sensor data from the item of footwear to a remote computing system,
receiving, from the remote computing system a predicted distribution of sensor data values to the item of footwear, and, during an operational phase:
monitoring further sensor data generated at the item of footwear, and, in the event that the further sensor data falls within the predicted distribution of sensor data values, controlling one or more vibration actuators to provide sensory stimulation in accordance with the program of therapy, training or movement assistance.

28. (canceled)
